(19) United States
(12) Patent Application Publication
(10) Pub. No.: US 2023/0405291 A1
(43) Pub. Date: Dec. 21, 2023

(54) EAR CATHETER AND INSERTION AID FOR A CATHETER

(71) Applicant: Auriventis GmbH, Loerrach (DE)

(72) Inventors: Holger Sudhoff, Bielefeld (DE); Maximilian Sudhoff, Bielefeld (DE); Aleksandar Obradovic, Loerrach (DE); Alle Obradovic, Loerrach (DE)

(73) Assignee: Auriventis GmbH, Loerrach (DE)

(21) Appl. No.: 18/037,490

(22) PCT Filed: Nov. 25, 2021
(86) PCT No.: PCT/EP2021/082967

§ 371 Date: May 17, 2023

(30) Foreign Application Priority Data
Nov. 27, 2020 (DE) 10 2020 131 572.4
Mar. 2, 2021 (DE) 10 2021 105 036.7

(51) Int. Cl. A61M 31/00; A61M 3/02; A61M 39/24

(52) U.S. Cl. CPC A61M 31/00; A61M 3/0283; A61M 3/0295; A61M 39/24; A61M 25/0074

(57) ABSTRACT

The invention relates to an ear catheter for insertion into the eustachian tube with a first tube as catheter tube (3) provided with at least one first lumen as injection duct (3A) for the application of a liquid/fluid, at least one second lumen as pressure relief lumen (3B) for pressure relief and a self-expandable sealing element (2) for the occlusion of the eustachian tube, as well as an insertion aid comprising a headpiece, a middle piece and an end piece, wherein the insertion aid consists of a biocompatible polymer, and with at least one separator line extending at least through each of the middle piece and the end piece, along which the insertion aid can be unfolded or split open. The insertion aid proposed by the invention can thus be removed from a catheter even if the catheter ends are blocked.

The invention relates to an ear catheter with a seal for the application of medications into the eustachian tube and to a catheter insertion aid, said aid comprising a headpiece, a middle piece and an end piece, with said insertion aid being made of a biocompatible polymer. The insertion aid is specifically designed to facilitate the placement of an ear catheter through a patient's nose into the middle ear.

There are numerous known diseases of the middle and inner ear that require local treatment. In the event a topical treatment has to be carried out, this can be accomplished on the one hand through the tympanic membrane and on the other hand via the eustachian tube. Treatment of the inner ear can also be administered through the middle ear region. For the treatment an ear catheter is frequently employed which is inserted through the nose, also to introduce medications. Nevertheless, administering drugs in liquid form particularly into the eustachian tube or middle ear is frequently considered less effective due to the fact that the drug may rather quickly drain through the eustachian tube into the nasopharynx.

A variety of infections, inflammations, but also the treatment of a hearing loss or Meniere's disease need a longer-term treatment to be performed, which may last for days or a few weeks. In this case, new medication, such as a corticosteroid for example, is to be introduced on a constant basis. This treatment is time-consuming and requires an ongoing and regular medical monitoring and care.

For this reason, it would be desirable to have available an ear catheter by means of which a drug could be administered into the middle ear and be kept there for an extended period of time. Such an application should possibly be non-invasive, i.e. without inflicting injury to the tympanic membrane. To achieve this, exclusively the natural access route must be followed which is via the nasopharynx and the eustachian tube.

In publication DE 10 2018 102 937 A1 a respective ear catheter is described that can be inserted into the eustachian tube via the nasopharynx and comprises an expandable balloon for sealing. An injection duct extends through the balloon which enables a drug to be applied into the eustachian tube distally of the balloon, the reflux of which into the nasopharynx is prevented by the balloon.

The disclosed ear catheter has several disadvantages. First of all, the catheter is not equipped with a pressure equalizing feature distal to the balloon that could balance out the excess pressure in the middle ear created by the application of a medium. Moreover, the known ear catheter does not have any markers by means of which the user could accurately determine the position of the inserted catheter. In addition, a balloon, which must function as sealing element as proposed, may even cause pressure marks. This can hardly be prevented by a pressure limitation feature which may possibly be provided to act on the balloon and/or the selection of a very adaptable balloon, since at least a certain pressure level must be applied in order to achieve a sealing effect of the balloon. Also, the handling of an ear catheter provided with an inflatable balloon is relatively complicated, because an additional device is always required to inflate (and deflate) the balloon.

Placement of an ear catheter through a patient's nose is not easy, especially if the catheter is made of very flexible material. For that reason, an insertion aid would be desirable here. Problematic is the use of an insertion aid, however, if a catheter has to be fixed in the middle ear or in the eustachian tube for a treatment, even over a longer period of time, because a device is usually arranged proximally which serves to connect the catheter to another device, for instance an infusion bottle or a device for applying pressure. In this case, the insertion aid cannot be simply pulled off proximally through the catheter tube, since another device or the connection for another device is located there. Removing the insertion aid in distal direction is equally impossible because the distal end of the catheter is secured in the target structure, such as in the eustachian tube for example.

It is therefore the objective of the present invention to provide an ear catheter equipped with a seal which does not have at least some of the disadvantages mentioned above, can be handled easily and, in particular, does not or only to a minor extent lead to irritation in the eustachian tube, even when left in place for a longer period of time.

It is, furthermore, the objective of the invention to provide an insertion aid by means of which a catheter, for example an ear catheter, can be inserted easily and quickly, for example through the nose into the eustachian tube of a patient, and which can be removed after placement of the catheter, at least in part, without the need of having to remove the catheter or any device connected thereto.

The objective is achieved by a device as defined by the independent claims 1 and 13. Further implementations or methods of the invention are defined in the dependent claims.

The objective is accomplished by an ear catheter proposed by the invention, which comprises a catheter tube having an injection lumen for the introduction of medication and a pressure relief lumen for pressure relief in the middle ear, as well as an expandable sealing element.

Such an ear catheter can be inserted non-invasively through the nose and nasopharynx into the eustachian tube and appropriately positioned therein such that the sealing element creates a fluid-tight seal between the eustachian tube and the pharynx after expansion has occurred. In this way, the sealing element thus serves to close off the connection between the middle ear area located distal to the sealing element and the nasopharynx proximal to the sealing element in such a manner that fluids introduced through the injection lumen terminating distally of the sealing element are effectively prevented from draining into the nasopharynx.

Within the context of the present invention the term “distal” refers to the portion of the device facing away from the user along the catheter or insertion aid, while “proximal” refers to the portion facing toward the user along the catheter or insertion aid. Further details on the orientational relationships can be seen in the figures.

The sealing element may be provided in the form of an inflatable balloon or, in a preferred embodiment, an expandable shield.

The injection duct extends continuously from the distal to the proximal end of the catheter tube and terminates distally after the sealing element.

The pressure relief lumen also starts at the proximal end of the catheter tube and ends distally after the sealing element. The exact location of the distal opening of the pressure relief lumen distal to the sealing element may vary, but with respect to functionality it is only important that the distal exit opening is also arranged distal to the sealing element.

Additional lumens may be provided in the catheter tube, for example a lumen to serve as an additional injection lumen or a lumen that, for example, serves to accommodate a removable support wire intended to stabilize the catheter tube during placement.

Aside from these lumens, the catheter tube also comprises at least one additional lumen serving as inflation lumen in the event that an inflatable balloon is provided as the sealing element.

For the catheter tube, customary materials as they are known in catheter manufacturing technology can be used. As a rule, these are medical-grade plastics, for example PVC, polyethylene, polypropylene and the like. The materials should be sufficiently flexible to enable the catheter to be inserted into the eustachian tube.

The balloon may also consist of customary materials as they are commonly employed for balloons in medical engineering.

The shield provided by the proposed invention comprises self-expandable support elements covered with a membrane, with the shield being reversibly transformed in its entirety from a compressed to an expanded state. If necessary, this allows a replacement of the shield.

The self-expandable support elements may be provided in the form of individual, unconnected support elements, for example, may consist of a plurality of annular or spoke-like segments. Preferably, however, the self-expandable support elements are at least partially interconnected and arranged and provided in the form of a framework, for example in the manner of a vascular support, a stent. Accordingly, the self-expandable support elements or the framework can be provided and made by laser cutting techniques from a tube or in the form of a braided structure.

Suitable materials used for the framework can be conventional shape memory materials, as they are also used for other self-expandable implants, such suitable materials being in particular spring steels, nitinol alloys or polymers. However, the materials for the shield framework should be non-absorbable so as not to endanger the stability of the shield.

Due to the fact that the shield of the ear catheter proposed by the invention exclusively has a sealing function and is not meant to keep a lumen open as other vascular supports do, only a minor radial force of the shield is required. High radial forces should on the contrary be avoided, as they may lead to the formation of pressure points and necrosis in the sensitive eustachian tube. The known materials enable the radial forces to be very precisely adjusted.

The self-expandable support elements, respectively the framework are covered by a membrane designed to be impermeable to liquids. Suitable materials for the membrane are appropriate liquid-impermeable biocompatible films, as they are also known from other implants provided with membranes—for example, stent grafts. Particularly suitable are various membranes made of plastic materials, for example made of PTFE or ePTFE.

The membrane must be securely arranged on the framework, for example by gluing, welding or using suitable clamping devices. Combinations of these techniques are conceivable. The membrane must be reliably attached to the framework, but this connection is not subjected to such stresses as, for example, in stent grafts, because the shield of the ear catheter will not be exposed to a constant flow of blood, but serves only to retain an applied liquid.

Preferably, the proximal portion of the membrane is connected to the distal end of the catheter in a fluid-tight manner, as described in detail hereinafter for the shield.

The shield is preferably of tapered or funnel-shaped design and has a proximal end having a first diameter and a distal end having a second diameter, with the second diameter being larger than the first diameter.

It is to be noted, however, that a variety of other shapes of the shield or shield framework are conceivable, which will be described in greater detail hereinafter on the basis of elaborate figures.

The shield is fixed with its proximal end to the distal end of the catheter tube. The first diameter of the shield is to be selected such that it enables an appropriate connection to be made with the catheter tube, that is, it must essentially match the diameter of the catheter tube.

An attachment aid may, preferably, be provided at the proximal shield end, for instance in the form of a tubular or hose-like extension that can be arranged and fixed within or around the distal catheter lumen and in this manner achieves and provides a fluid-tight connection between the shield and catheter tube. The diameter of the attachment aid is to be selected such that it fits the diameter of the catheter, with the outer diameter of the attachment aid being in particular equal to the inner diameter of the catheter or vice versa.

Other connection configurations between shield and catheter are conceivable. To name just a few of the possibilities, these configurations particularly encompass screw connections, snap-in connections, form-fit connections, adhesive connections or injection-molded connections.

It must be ensured that the attachment location where shield and catheter tube are joined must not impair the expansion or compression of the shield produced by the expansion device, which will be described in more detail later. For this reason, an attachment of the shield to the outside of the catheter tube should rather be avoided, which is also true for any attachment configuration of the shield that results in an edge forming on the outside of the catheter.

Preferably, the shield is mounted and attached to the inside of the distal end of the catheter tube or terminating at the end of the catheter, i.e. at the distal outlet opening of the catheter tube, in such a way that the lumens of the outlet openings, in particular those of the injection lumen and the pressure relief lumen, are not occluded/obstructed, and the application of a liquid and the discharge of excess pressure are not impaired. The same applies analogously to the inflation lumen in the event a balloon is provided as sealing element.

In an alternative embodiment of the variant comprising a shield as a sealing element, the lumens in an extended section of the catheter tube may also extend distally beyond the distal end of the shield. With a view to preventing injury, particularly in this embodiment and the embodiment comprising a balloon, an atraumatic tip is provided at the distal end of the catheter tube extended in this way.

The second diameter of the shield as well as, respectively, the normative diameter of the balloon must be selected such that they correspond at least to the diameter of the eustachian tube at the point where they create the fluid-tight seal. Preferably, the second diameter of the shield or, respectively, the normative diameter of the balloon are to be selected so as to be slightly larger than the relevant diameter of the eustachian tube.

The construction of the shield comprising framework and membrane is such that the expansion device, which will be described in more detail hereinafter, can be easily both retracted in proximal direction away from the compressed shield and pushed forward in distal direction over the expanded shield.

For this purpose, it is advisable to arrange for the shield to be of funnel-shaped or conical/tapered design, i.e. to provide a structure that widens from proximal to distal. At least, the shield should be provided to include an appropriately widening area at the end that connects to the catheter tube.

In a state with no external constraints being exerted neither from the expansion device nor from, for example, the eustachian tube, the shape of the shield in the freely expanded state can be determined by the membrane and/or the framework. This means, either the membrane limits the maximum possible expansion of the shield at the respective location, or the framework, resp., supporting elements do.

The shield and the catheter tube are interconnected in a liquid-tight manner which ensures that a liquid applied through the catheter tube cannot escape proximally by passing between the catheter tube and the expanded shield.

With the aid of the expansion device, the shield can be reversibly changed from a compressed to an expanded state. For this purpose, the expansion device—as well as the catheter tube is provided to have a tubular form and encloses the catheter tube in a most largely form-fitting manner, with a liquid-tight seal between the expansion device and the catheter tube not being required. The tightness of the ear catheter to liquid is exclusively brought about by the shield.

With respect to the catheter tube, the expansion device is arranged to be movable at least in the axial direction enabling it to be moved along the catheter tube from proximal to distal or in the reverse direction. Preferably, the expansion device reaches maximally from the proximal end of the catheter tube to the point where the shield is attached to the catheter tube, with the said device being displaceable along the catheter tube to beyond the distal end of the shield.

Displacing the expansion device in distal direction results in the shield being compressed, namely when the expansion device is moved over the shield which is secured at the distal end of the catheter tube. On the other hand, when the expansion device is retracted off the shield and moved in proximal direction this will result in the shield being expanded and permitted to open up due to the fact that the expansion device no longer exerts any external constraint on the shield.

For better handling, an optional operating element is arranged at the proximal end of the expansion device by means of which the expansion device can be shifted along the catheter.

The tube of the ear catheter should have a length of between approximately 15 cm and 30 cm. The length should be sufficient to insert the catheter through a nostril and the nasopharynx into the eustachian tube, with the proximal end positioned in the patient's cheek area or, in an alternative embodiment, being fixable behind the relevant ear of the patient.

At its proximal end, the ear catheter, resp. the injection lumen is provided with a connection device for common syringes allowing the ear catheter to be filled with pressurized medium and medication solution to be introduced into the inner ear. Expediently, the connection device is a Luer-Lock system as it is commonly used in the medical field, to which various syringes can be coupled via appropriate equipment.

Accordingly, the pressure relief lumen at the proximal end of the catheter can also be provided with a connection device, but said lumen may also terminate without a special connection device. Expediently, the connection device is the Luer-Lock system as commonly used for medical purposes, to which various syringes can be coupled via suitable equipment.

Preferably, the expansion device can be releasably secured at the proximal end of the catheter tube when the shield is in expanded state to prevent inadvertent compression of the shield. For this purpose, a releasable connection option is preferably provided between the operating element of the expansion device and the Luer-Lock system. Conceivable here are simple latching elements which are provided compatible with each other on the Luer-Lock system and the operating element. Other latching devices are conceivable, and a wide variety of solutions are known to those skilled in the art.

To prevent an inadvertent expansion action caused by slipping of the expansion device in proximal direction during placement of the ear catheter through the nose and nasopharynx, a spacer element may be provided between the connecting device at the proximal end of the ear catheter and the operating aid of the expansion device, said spacer element being conveniently provided in the form of a slotted tube, so that it can be pulled off and put back on.

It shall be understood that the ear catheter or the injection lumen is provided with a closure device that prevents the applied medication from flowing back after it has been filled in. To this end, the injection lumen is provided with a closure device, such as a one-way valve, at either its distal or its proximal end, said device ruling out that the injected drug can drain through the injection lumen. In the case of a one-way valve, the pressure exerted by the injection process causes the valve outlet to open which then shuts off again.

Also the pressure relief lumen is provided with a closure device, such as a one-way valve, at either its distal or its proximal end, said device preventing the injected medication to drain through the pressure relief lumen. In the case of a one-way valve, the pressure exerted causes the valve outlet to open which then shuts off again.

The closure device can be provided as a simple self-closing valve.

It is advantageous to equip the catheter with a handling aid for control during placement. It is, moreover, considered advantageous to provide a bent distal end on the catheter with a view to facilitating control and positioning of the catheter into and within the eustachian tube. Such a bent end is called a pigtail on catheters and guidewires and is widely in use.

The ear catheter proposed by the invention is inserted non-invasively through the nose and nasopharynx into the eustachian tube. The insertion can be monitored by ear microscopy. As soon as the expandable sealing element, that is, the shield or the balloon, has reached the part of the eustachian tube to be closed off, it is expanded by actuating the expansion device.

However, to assist in monitoring the position of the catheter or sealing element, markings may be arranged at the distal end of the catheter, but in any case, proximal to the sealing element. Such markings, for short hereinafter referred to as markers, can be provided sequentially and visualized endoscopically by the practitioner during placement. It is considered advantageous to apply several markers that for instance identify a first noncritical area of catheter advance, for example using a green marker, a second area of increased attention, for example denoted by a yellow marker, and a third critical area, for example using a red marker. In this context, the terms “noncritical”, “increased attention”, and “critical” refer to a progressive penetration of the catheter tip into the inner ear and thus alert to an increasingly critical proximity to sensitive areas.

The injection lumen terminates distally of the sealing element. At the proximal end, it preferably is provided with a Luer-Lock connector for a syringe by means of which a drug can be applied to the middle ear region.

The inventive ear catheter is particularly suitable for the application of antibiotic solutions into the middle ear region, which is an expedient approach towards combating persistent bacterial infections. Expediently, the standard treatment of a hearing loss, during which corticosteroids are applied, can also be administered with the aid of such an ear catheter. The standard therapy approach requires that such a corticosteroid be applied for a prolonged period of time, approximately two weeks as a rule, with a daily reapplication being necessary since the drug drains off into the nasopharynx. In this case, the usual duration of treatment is 14 days. The ear catheter proposed by the invention enables the treatment to be much more intensive and shorter since it ensures the constant presence of the medication. This reduces the duration of treatment considerably, possibly to just a few days. The catheter is especially suitable for retaining the drug in the middle ear for a longer period of time, in particular even several days.

Furthermore, anti-inflammatory drugs can also be introduced through the ear catheter.

Placement of the ear catheter is done through one of the nostrils, if thought expedient or necessary by making use of the insertion aid proposed by the invention, and, after expansion of the shield and application of the drug, the catheter can be fixed on the cheek or alternatively behind the ear using an appropriate medical strip.

During treatment, the ear catheter remains in the eustachian tube and in this way retains the applied medication in the eustachian tube area thus making sure it cannot flow back into the nasopharynx. The patient thus remains mobile so that a longer stay in a clinic is not necessary, regular medical monitoring will be sufficient. Ear microscopy can be employed for monitoring purposes.

When treatment has been completed and compression of the sealing element taken place, the ear catheter can be easily removed.

The ear catheter also allows treatment of diseases of the inner ear, since the medication can usually pass through the membrane of the round window to the inner ear. In this case, a diffusion process is involved here.

Possible indications include, inter alia, intratympanic steroid administration in case of a hearing loss, intratympanic steroid administration for hydropic ear disease (Meniere's), intratympanic gentamicin administration for hydropic ear disease, intratympanic contrast agent administration for suspected hydropic ear disease, intratympanic application for gene therapy (CRISPR/Cas9), or intratympanic topical antibiosis. Use of the catheter for other indications is at the discretion of the treating physician.

A range of substances that can be applied in this way include, for example, dexamethasone, methylprednisolone, gentamycin, neomycin, growth factors, BDNF, neurothrophin-3, N-acetyl cysteine, dextran, rhodamine, or even gadolinium salt. The application of other substances is at the discretion of the treating physician.

Advantageously, the ear catheter is positioned by means of a special insertion aid. An insertion aid proposed by the invention and suitable, inter alia, for use with an ear catheter comprises three sections, namely a headpiece, a middle piece and an end piece, with a separator line extending in the longitudinal direction of the respective section along at least some of the sections, allowing the respective sections of the insertion aid to be opened longitudinally.

In other words, the essential idea of the invention with respect to the insertion aid is to provide an insertion aid that can be at least partially removed from a catheter that has been placed in position, in that one or several longitudinally extending separator lines are arranged in some or all of the insertion aid sections. The separator lines may extend over individual, several or all sections of the insertion aid and they can be of continuous configuration, i.e. a continuous separator line over several or all sections is also conceivable, or the separator lines in the individual sections can have a different design and/or be arranged in different positions.

A separator line within the meaning of the invention may be a line of weakness where the strength of the material at that particular location of the insertion aid is comparatively lower. For example, such a line of weakness can be produced by laser treatment but is more conveniently generated mechanically by incisions or perforation lines that facilitate tearing along this line.

Such incisions may extend, for example, across 40 to 70% of the wall thickness of the insertion aid in the relevant section. In this case, full separation only takes place by the action of the user.

However, a separator line in the sense of the invention can also be an already existing opening where the material has already been completely cut through at the relevant position, for example in the case of a multi-part production of the respective part. In this case, it is not the user or operator who completes full separation. Rather, the individual parts are joined by various connector elements to be described later herein.

The insertion aid is essentially made of a relatively stiff material, usually a polymer. Polymers suitable for use in the field of medical engineering are known; thermoplastics and thermoplastic elastomers, such as polyether block amides (Pebax), are particularly suitable. A certain degree of flexibility makes catheter insertion easier, but too much flexibility means a loss of guiding capability.

In a first preferred embodiment, the insertion aid is provided for use with an ear catheter. The headpiece of the insertion aid is bent or angled to allow the easiest possible placement in the eustachian tube. The middle piece and the end piece are essentially straight.

These three sections have common dimensions, for example, about 2.5 cm for the headpiece and about 14 cm for the middle piece. The headpiece and middle piece are inserted completely into the body via the patient's nose, while the end piece, which for example has a length of 15 cm, remains outside the body.

It goes without saying, that the material of the insertion aid consists of a biocompatible material and it is recommendable for this material to be transparent.

The headpiece is angled in the usual way with respect to the configuration of the middle piece, for example by an angle ranging between 30° and 80°, in particular appr. 70°.

In this first embodiment of the insertion aid, two separator lines run the length of both the headpiece and the middle piece, allowing this portion of the insertion aid to be torn open. The end piece itself is split thus forming two ends that can be grasped by the user/operator to pull the insertion aid out of the middle ear, eustachian tube and nose when the catheter is in place.

The separator lines of the headpiece and the middle piece continue proximally to form the separator lines of the two halves of the end piece, so that the user will be able to tear open and remove the entire insertion aid via the free ends, with the used insertion aid then being disposed of.

It should be noted that “distal” in this context refers to the part of the insertion aid pointing toward the middle ear, while “proximal” refers to the part pointing away from the middle ear.

It has been found that in individual cases the insertion of the ear catheter by means of the insertion aid according to the first embodiment may lead to strains acting on the area of transition between the middle piece and the end piece in such a way that the separator lines begin to tear open. To avoid such a premature tearing, it is expedient to provide the proximal end of the middle piece with a safeguard that prevents such tearing. A suitable safeguard is, for example, a ring of adhesive tape which is torn off by the user before the insertion aid is removed. For this purpose, it conveniently has a free, non-adhesive end that facilitates removal.

In practice, having placed the catheter in position, the user takes hold of the two free ends of the end piece of the insertion aid, then pulls the middle piece and the headpiece along the catheter and out of the patient's body thus tearing open the connected parts along the separator lines, first on the middle piece and then on the headpiece. This separates the insertion aid from the catheter.

Also in the context of the second preferred embodiment, the insertion aid can be used with an ear catheter. For this purpose, the headpiece can have a bent or angled configuration as described hereinbefore to allow the easiest possible placement of the insertion aid in the eustachian tube. The middle piece and the end piece are essentially straight.

Other details that have been stated in the first embodiment with respect to dimensions and preferred materials of the insertion aid apply here as well.

The second preferred embodiment differs from the first preferred embodiment primarily with regard to the way the insertion aid is separated from the catheter. In the second embodiment, separating the insertion aid from the catheter is in particular brought about by unfolding the insertion aid.

As provided by this second embodiment the insertion aid is of folded design and preferably consist of two parts, i.e. it can be separated lengthwise into two halves. The separator lines in this case are provided in the form of continuous slots as previously described here. Preferably, the two halves are provided as mirror-image identical halves. It is, however, also conceivable that the insertion aid does not consist of such identical mirror-image halves, but is composed of corresponding thirds or quarters or in other proportions. Nevertheless, preference is given to identical mirror-image halves.

The halves are preferably joined together permanently at least at one point so that they do not fall apart into two separate parts when unfolded along the separator lines. An appropriate connecting element can be provided, for example, in the form of a hinge, said hinge being preferably arranged in the area of the end piece and serves to put the two halves together. The hinge can already be provided between the two halves during the manufacture of the operating aid, for example in an appropriate injection molding process, or it can be designed for joining the two halves after they have been manufactured.

In embodiments comprising a plurality of longitudinal members as previously described, additional hinges are suitably provided between the longitudinal members.

In lieu of the hinges, other connecting elements may also be provided to join the longitudinal parts of the insertion aid. Such connecting elements do not necessarily have to create a permanent bond; it is sufficient if the individual longitudinal parts are reliably held together during placement of the catheter.

Equally useful is a combination comprising hinges and other connecting members. A reliable connection of the insertion aid can be achieved, for example, by at least one hinge located on the end piece, and further connecting elements to be provided for support on the middle and end piece or also on the headpiece.

These further connecting elements can be, for example, latching or snap-in elements, the counterparts of which are located on the respective longitudinal parts which have to be put together. In this case, the further connecting elements preferably form part of the insertion aid.

However, further connecting elements are also conceivable, for example, in the form of rings which are provided on the relevant longitudinal parts of the insertion aid and which can be released to unfold the insertion aid, for example at predetermined breaking points arranged for this purpose. Further connecting elements may also be provided in the form of the safeguards previously described.

It is also conceivable for the second embodiment of the insertion aid to be provided in the form of a reusable product due to the non-destructive unfolding mechanism of the essential parts. Accordingly, a more solid design approach can be pursued, particularly in the construction of the end piece, so that a particularly comfortable feel is achieved, for example, through the use of specially shaped grip areas. Likewise, reusable connecting elements may be provided in the end piece, for example in the form of latches that enable the folded or joined end piece to be repeatedly opened and closed.

In accordance with an alternative design of the second embodiment, only the middle and end pieces are provided so as to be foldable. The headpiece, on the other hand, is provided in a one-piece tubular form and can be connected at its proximal end via a coupling element to the distal end of the middle piece.

The coupling element can be provided in the form of a thread, for example. In this case, the headpiece has a male or female thread with a suitable counterpart being arranged on the middle piece. It is also feasible for the coupling element to be arranged in the form of a separate component, i.e. as a threaded element into which the head and middle pieces are fitted, with in this case as well the arrangement of suitably matching male and female threads being selected by the skilled person to meet the respective requirements. Preferred is an embodiment in which the headpiece and middle piece have a male thread and these male threads being screwed into the female thread of the coupling element.

Other coupling elements, for example in the form of, in particular, force-fit snap-in or latching elements, are conceivable.

In this case, the coupling element additionally serves as a connecting element in that it joins the two parts of the middle piece and the end piece connected to it.

In the alternative second embodiment of the insertion aid, the headpiece thus remains on the catheter after removal of the middle and end pieces and can be used, for example, as a fixation aid after having been shifted toward the proximal end of the catheter, e.g. to secure the proximal end of the catheter behind the patient's ear.

The alternative second embodiment of the insertion aid also offers the advantage that the headpiece is replaceable. By providing an appropriate set, a multitude of headpieces of different angles could then be added to the basic element of such an insertion aid, namely to the unit consisting of a middle and end piece. From the assortment of different headpieces available, the user can then select the headpiece that is best suited for the particular assignment to be carried out. Most diverse variations are conceivable here with regard to the angle of the headpiece, the length of the headpiece or also the materials, which may range from particularly stable to flexible.

Accordingly, headpieces may be provided in such a set that do not necessarily involve treatments of the ear and thus are also suitable for use with other catheters.

Thus, the alternative second embodiment of the insertion aid may also be provided as a combination product comprising a reusable basic element with interchangeable and disposable headpieces.

Alternatively, the second embodiment may additionally comprise a tubular sheath at the head and/or middle sections that closely embraces these sections. This sheath provides additional support to the two-piece head and/or middle pieces by firmly holding the two halves of these components together. The sheath can be provided in one piece or consist of several pieces and can be attached at different points of the head and/or middle piece or it may also be provided exclusively in the form of a ring, i.e. may not be a longer or continuous tube.

A perforation line may be provided on the sheath for opening and removing purposes; however, such a perforation is mainly arranged to improve ease of use. In the absence of a relevant perforation, the sheath may alternatively be removed by the user by making a longitudinal cut or by tearing it open. The material of the sheath can be selected to suit the respective purpose.

Since the sheath is also inserted during treatment, low-friction materials are particularly advantageous here. Persons skilled in the art are familiar with a wide range of materials, such as various plastics, for example PTFE or ePTFE.

As explained hereinbefore, the invention relates in particular to the use of an insertion aid for the placement of an ear catheter. Moreover, the invention relates to a combination of an insertion aid and an ear catheter that are dimensionally matched to each other. In this context, the ear catheter can be integrated into the insertion aid ready for insertion.

Even though the invention was previously described primarily in connection with ear catheters and the use thereof for the placement a catheter in the eustachian tube, the application of the inventive insertion aid shall by no means be restricted to such areas. Rather, the use of the insertion aid proposed by the invention is in fact useful wherever removal of an insertion aid after successful placement can no longer take place without difficulty in a distal or proximal direction along the catheter for the reasons described above.

Therefore, possible uses and areas of application of the insertion aid proposed by the invention are not limited solely to those that relate to ear catheters in the nasopharynx. Moreover, the inventive insertion aid can in fact be suitably adapted to the respective purpose or place of use in particular by appropriately designing its individual sections, such as headpiece, middle piece and end piece.

To this end, these sections can be modified, for example, in their dimensions such as length and diameter or their bending configuration, or e.g. the selection of materials to suit the flexibility requirements of the respective place of application.

The invention as well as the technical environment are described hereunder in sufficient detail on the basis of the figures. It is to be noted that the figures show an especially preferred embodiment variant of the invention. However, the invention shall not be deemed as being limited to the embodiment variant shown. To the extent it is technically expedient, the invention comprises, in particular, any optional combinations of the technical features that are stated in the claims or in the description as being relevant to the invention.

CLARIFICATION OF THE INVENTION IS PROVIDED BY THE FOLLOWING FIGURES WHERE

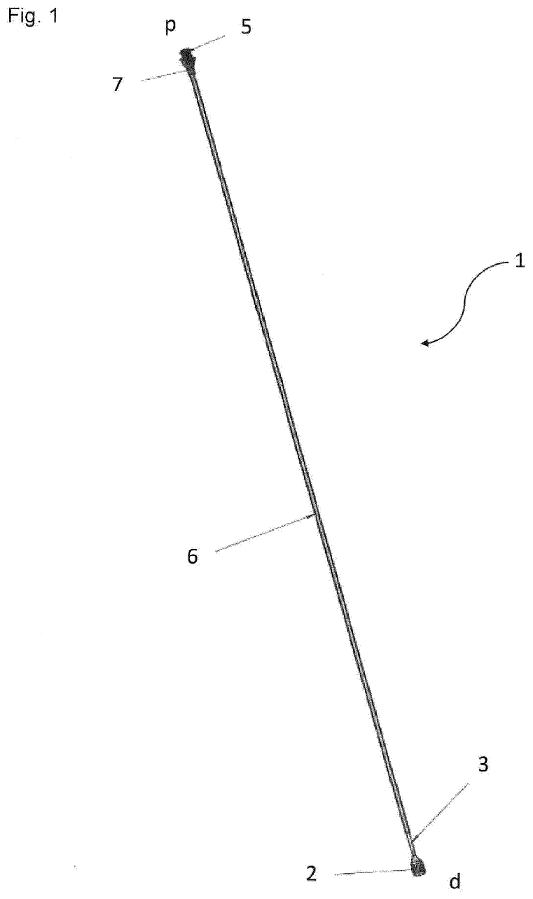

FIG. 1 is a general view of a first preferred embodiment of the ear catheter according to the invention with a shield as sealing element,

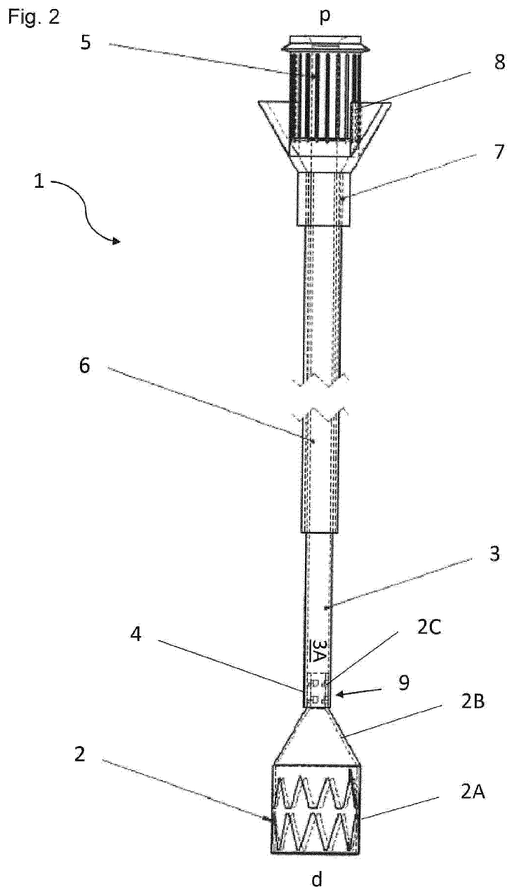

FIG. 2 shows details of the proximal and distal sections of the preferred embodiment according to FIG. 1,

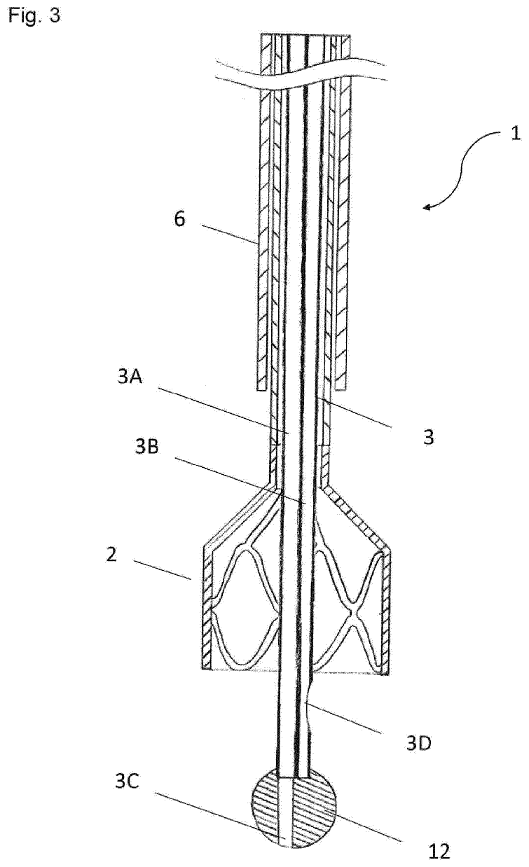

FIG. 3 illustrates further details of the distal section of another preferred embodiment,

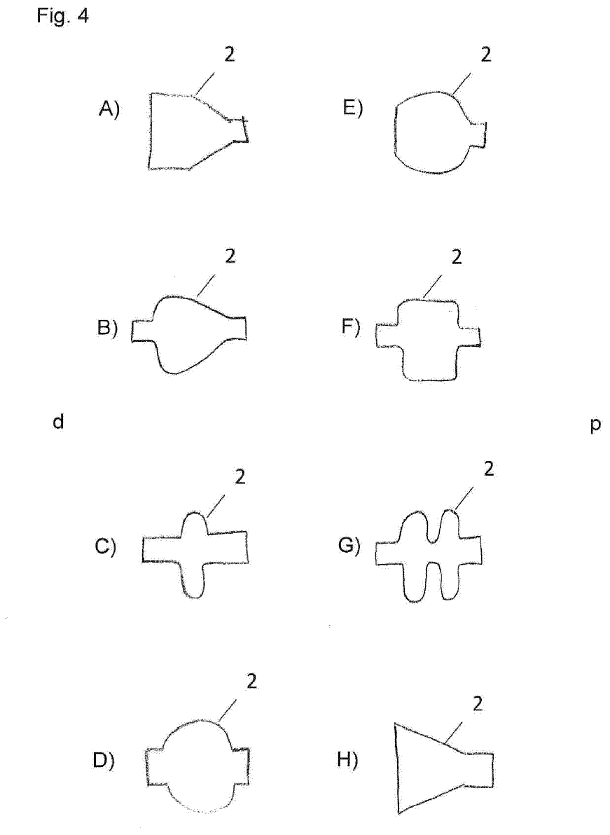

FIG. 4 shows various outlines of possible shield configurations,

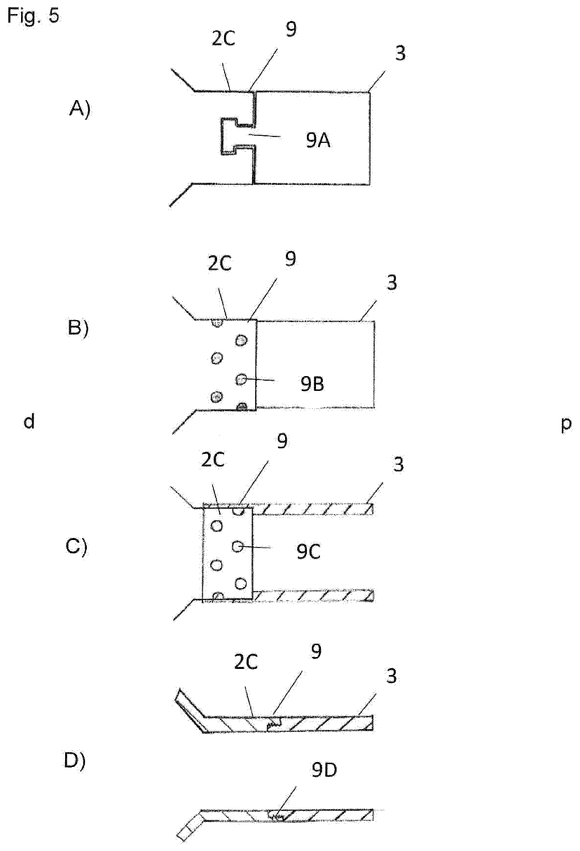

FIG. 5 shows various ways of connecting the proximal shield section to the catheter tube,

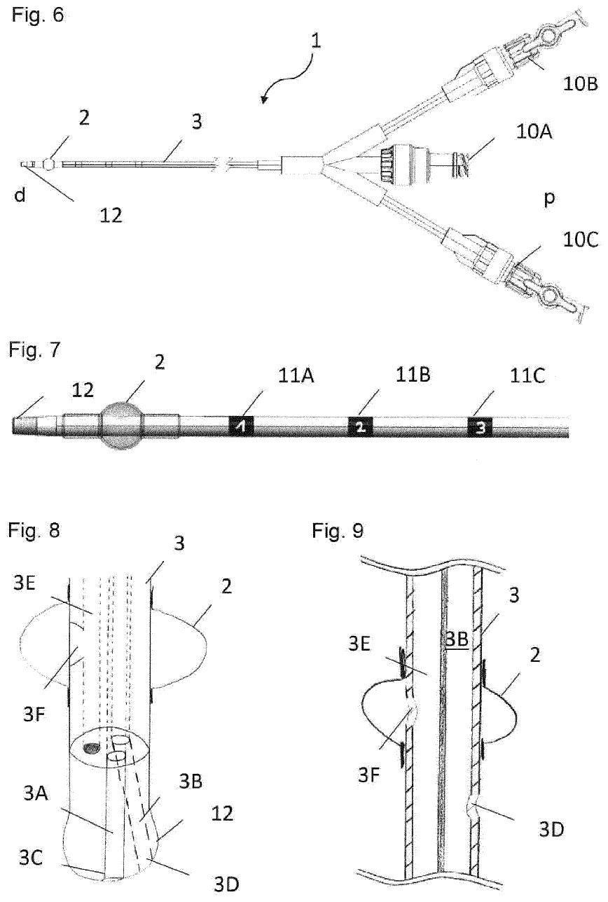

FIG. 6 is an overview of a preferred embodiment with a balloon as sealing element,

FIG. 7 illustrates details of the proximal end of the embodiment according to FIG. 6,

FIG. 8 includes further details of the proximal end of the embodiment according to FIG. 7,

FIG. 9 includes further details of the proximal end of the embodiment according to FIG. 7 as a variant,

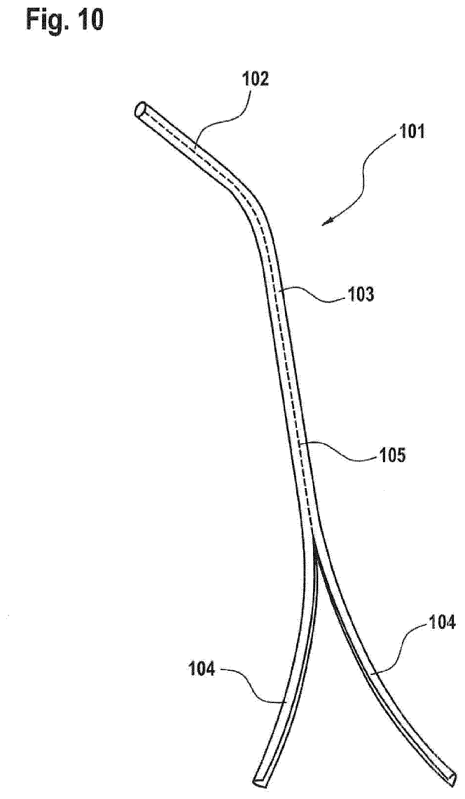

FIG. 10 shows a first embodiment of the insertion aid proposed by the invention,

FIG. 11 depicts the principle of use and removal of the first embodiment of the insertion aid,

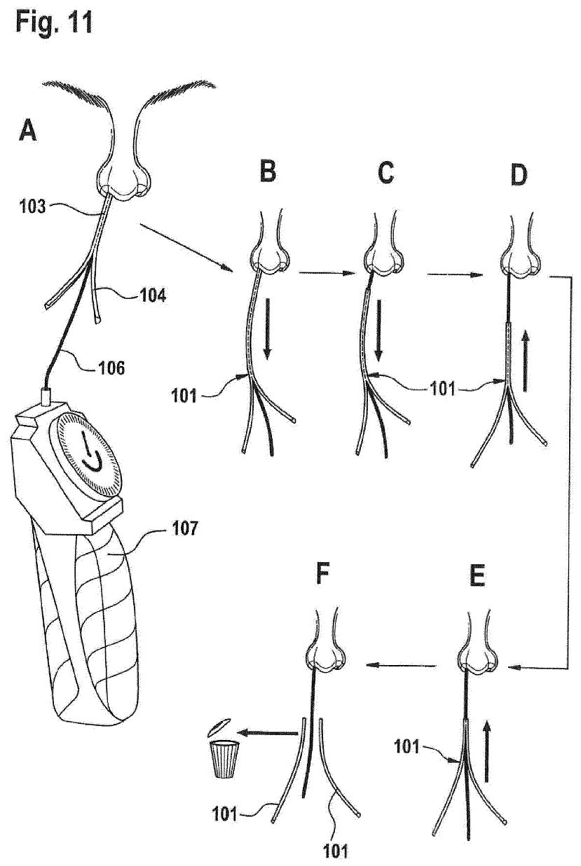

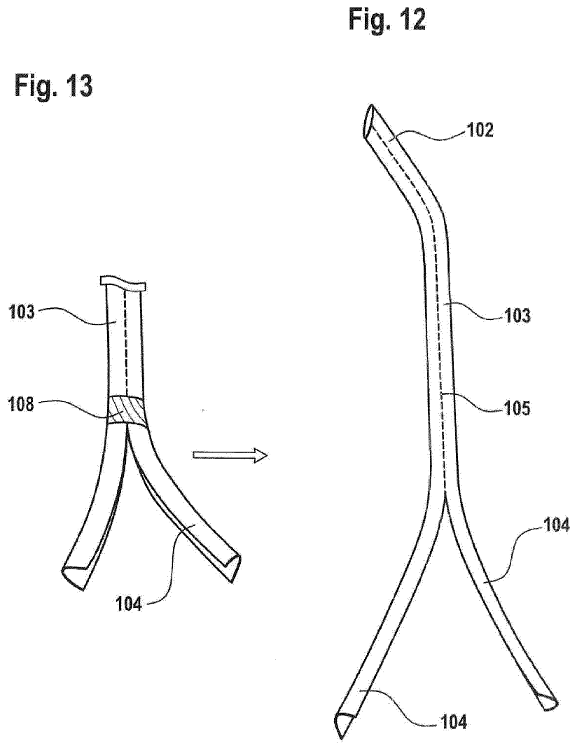

FIG. 12 shows the extension of a separator line of the first embodiment,

FIG. 13 shows a first embodiment of the inventive insertion aid provided with safeguard,

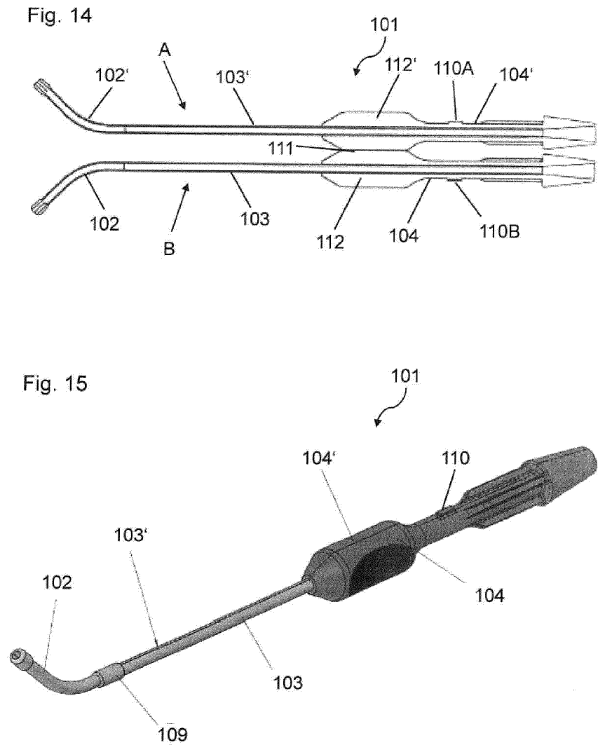

FIG. 14 is a second embodiment of the insertion aid according to the invention in the unfolded state,

FIG. 15 is a perspective view of an alternative design of the second embodiment of the insertion aid according to the invention,

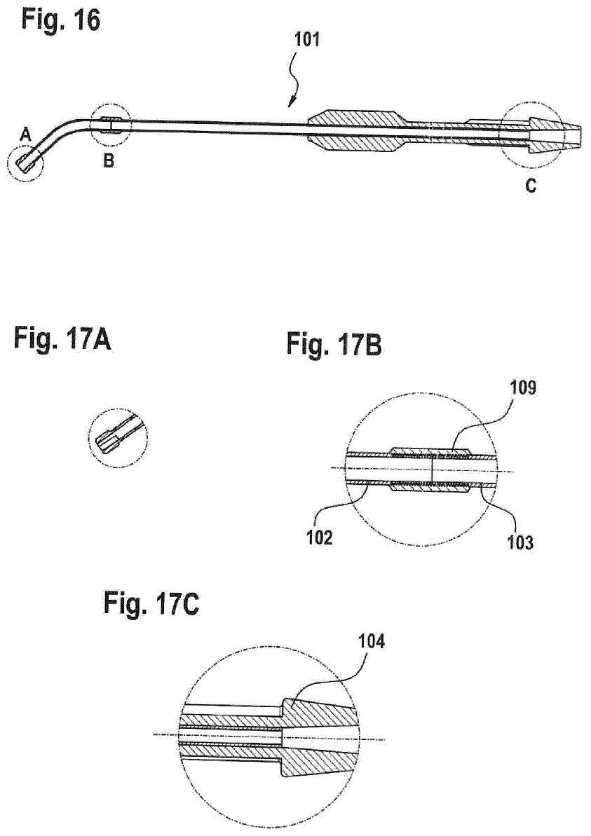

FIG. 16 is a top view of the inside of one half of the insertion aid according to FIG. 14,

FIG. 17 shows detailed views of FIG. 16,

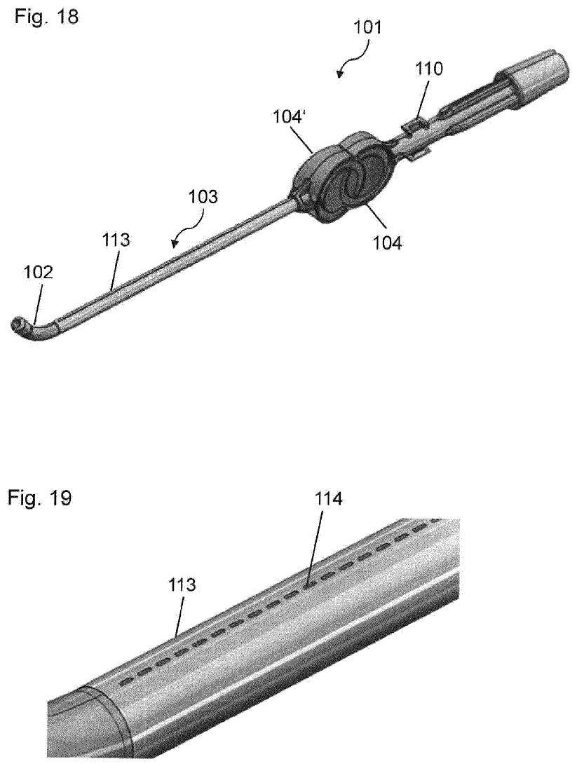

FIG. 18 is a perspective view according to FIG. 6 provided with sheath,

FIG. 19 illustrates a section detail of the perspective view according to FIG. 18.

The ear catheter 1 proposed by the invention as shown in FIG. 1 comprises a catheter tube 3, at the distal end d of which a self-expandable shield 2 is arranged. At the proximal end p of the catheter tube 3 there is a Luer-Lock connector 5 for a conventional syringe, by means of which the medication to be applied can be inserted through the injection lumen 3A. Moreover, an additional connection for the pressure relief lumen (not shown) can be arranged at the proximal end. In this embodiment, the only lumen of the catheter 3 is the injection lumen 3A; however, an embodiment is preferred in which the catheter comprises another lumen for pressure relief purposes in addition to the injection lumen 3A, as shown in FIG. 3 hereinbelow.

The ear catheter 1 has an overall length ranging between about 15 cm and 30 cm with an outer diameter of the catheter tube 3 of preferably 2 to 3 mm and an inner diameter of the injection lumen of about 1 mm.

A tubular expansion device 6, which is arranged in a form-fitting or frictionally engaged manner around the catheter tube 3, is used to expand or compress the shield 2, with said expansion device 6, same as the catheter tube, preferably being made of plastic.

An operating aid 7 is provided at the proximal end p of the expansion device 6, which simplifies the handling of the expansion device 6 in that it enables the expansion device 6 to be safely gripped and thus safely controlled.

By means of expansion device 6 the expansion or compression of the shield 2 is brought about. With the expansion device 6 being pushed in distal direction d over the shield and left there, this results in the shield 2 to be compressed; if the expansion device 6 is pushed in proximal direction p off the shield 2 and left there, this causes shield 2 to be in the expanded state.

To prevent an inadvertent expansion of the shield 2 caused by a slipping of the expansion device 6 during placement of the ear catheter 1 through the nose and nasopharynx, a spacer element may be provided between Luer-Lock 5 at the proximal end p of the ear catheter 1 and the operating aid 7 of the expansion device 6, said spacer element being expediently provided in the form of a slotted tube, so that it can be pulled off and put back on (not shown).

FIG. 2 shows the details of the distal end d and the proximal end p of the ear catheter 1. The self-expanding shield 2 is provided at the distal end d of the ear catheter 1, said shield being preferably composed of three sections 2 A, B, C, i.e. a distal section 2 A of large diameter which has a cylindrical shape over a certain length without appreciable tapering and which essentially represents that part of the shield 2 which makes fluid-tight contact with the wall of the eustachian tube. An optional tubular proximal section 2 C is provided for safely securing the shield 2 within the distal lumen of the catheter tube 3.

However, it should be made sure that the open diameter of the catheter tube 3, which includes the injection lumen 3A and the pressure relief lumen (not shown) is reduced only insignificantly so as not to interfere with fluid application and pressure equalization. Between the proximal section 2 C and the distal section 2 A, a middle section 2 B is arranged, which is of funnel or cone shape and widens from the smaller diameter of section 2 C toward the larger diameter of section 2 A.

The configuration of shield 2, which means the proportions of the self-expanding support elements or framework on the one hand and of the membrane on the other, can vary within the sections 2 A to C. Preferably, to ensure the most effective seal possible, the membrane is of one-piece design and extends over all sections 2 A to C.

To make sure the tightest possible seal is brought about between the shield 2 and the wall of the eustachian tube, a cylindrical section 2 A may be provided distally over a certain length. Aside from the membrane, this section also comprises a multitude of self-expanding support elements, which are preferably arranged as meandering ring segments and may be provided in the form of an interconnected framework or exist as separate support elements. However, a distal section 2 A is optional, and embodiments are also conceivable in which the distal end of the tapered section 2 B already constitutes the distal end of the shield 2.

Sections 2 A to C can be supported in whole or in part by self-expanding support elements or by means of an interconnected framework. The structure and design may vary in each of the sections 2 A to C. It is, however, of importance at the distal end d of the shield 2 that an adequate but not too high radial force is exerted there, which ensures an as tight a seal as possible between the shield 2 and the eustachian tube, but at the same time does not lead to pressure points and necrosis. In this respect, the distal end of section 2 B and/or the optional section 2 A are to be designed accordingly. Otherwise, the tapered section 2 B does not need to be subjected to higher radial forces, since its main purpose is to prevent the drug from flowing back from the eustachian tube into the nasopharynx. Accordingly and in case a distal section 2 A has been provided, section 2 B would in principle be conceivable without a framework structure or relevant support elements. The same holds true for section 2 C, which ultimately serves solely to secure the shield 2 in the catheter tube 3, which is also conceivable solely by appropriately connecting the membrane to the catheter tube 3.

Conceivable are embodiments in which the complete shield 2 is formed by an inner framework and/or suitable support elements, with a membrane being connected thereto. Without any inventive effort, relevant combinations of sections 2 A to C of the shield, which besides the membrane also comprise a framework and/or support elements, can be devised by skilled persons to satisfy the respective requirements.

The expansion device 6, which is provided in a tubular form around the catheter tube 3, optionally comprises an operating aid 7 at its proximal end, which facilitates sliding the expansion device 6 forward and backward. In a preferred embodiment, a latching device 8 may be provided to releasably connect the operating aid 7 to the Luer-Lock system 5 to prevent a displacement of the operating aid 7 when the shield 2 is expanded and, in particular, to prevent the shield 2 from being compressed.

The expansion device 6 is preferably provided in the form of a hose or tube. Moreover, conceivable are also embodiments in which the expansion device 6 is provided, at least partially, with slots, for example to enhance the flexibility of ear catheter 1.

The operating aid 7 can be provided in the form of a ring, although slotted embodiments or retaining elements arranged at only certain points on the expansion device 6 are conceivable as well.

To prevent backflow of the applied liquid through the injection lumen 3A, a one-way valve 4 is preferably located in its distal end. A corresponding valve is provided for the pressure relief lumen (not shown).

After the placement of the catheter and expansion of the shield, the treatment proceeds by filling the medication into the middle ear via a syringe and with the drug remaining there for the required time. Filling of medication into the middle ear is monitored by ear microscopy. Preferably, the procedure is performed under local anesthesia, but in special cases general anesthesia may be recommendable. After the medication has been administered, the proximal part of the application catheter is stuck to the cheek with a medical strip. The sealing element remains in place in the eustachian tube and prevents the injected solution from draining off. At the end of the treatment, the sealing element is compressed and the catheter is withdrawn.

FIG. 3 shows another preferred embodiment of the ear catheter 1 identifying the injection lumen 3A and the pressure relief lumen 3B. At the distal end, the catheter tube 3 terminates in an atraumatically formed tip 12, wherein the injection lumen 3A terminates in opening 3C distally of the atraumatically formed tip 12, with the pressure relief lumen 3B terminating proximally of the atraumatic tip 12 with an opening 3D. Other than the embodiment illustrated in FIG. 2, at least part of the catheter tube 3 with injection lumen 3A and pressure relief lumen 3B extends through the shield 2 and terminates distally of the shield 2.

In the illustrations A to H in FIG. 4 various possible configurations of the shield 2 are depicted in outline representations, with in said illustrations the distal end being located on the left and the proximal end on the right in each case. The outlines respectively reflect the combination of framework and membrane, i.e. the configuration of the shield 2 in each individual case will essentially be determined by the framework, the membrane or the combination thereof.

FIG. 5 shows details of various connectors 9 by means of which the proximal shield section 2C and the catheter tube 3 can be interconnected. FIG. 5A shows a connector 9 including latching elements 9A, said connector being preferably be bonded by injection molding. In FIG. 5B an adhesive connector 9 is shown, and in this case the catheter tube 3 extends into and is adhesively bonded to the proximal end of the shield 2C. To further stabilize the adhesive connector 9, the proximal shield end 2C may include punched-out holes 9B. Alternatively, this connection can also be made as a straightforward form fit. FIG. 5C shows a form-fit connector 9 with optionally arranged additional retaining elements 9C, which increase the frictional resistance and thus additionally prevent the shield from sliding out. The catheter tube 3 in this embodiment surrounds the distal shield end 2C, and this connector may also be provided as an adhesive connection. FIG. 5D shows a connector 9 provided with a threaded connection 9D, with the male thread to be part of the distal shield end 2C and the female thread to be part of the catheter tube 3, or vice versa.

FIG. 6 is a complete view of the preferred embodiment of the ear catheter 1 including a balloon as sealing element 2. Three connections are located at the proximal end of the catheter 3, i.e. connection 10C for inflation of the balloon, connection 10A accommodating a syringe for the injection lumen, and connection 10B for pressure balancing via the pressure relief lumen. An atraumatic tip 12 is provided at the distal end of the catheter 1.

In FIG. 7 a detailed view of the distal catheter section is illustrated including atraumatic tip 12, the balloon 2 located proximal to it and three markers arranged proximal to the balloon, said markers serving to endoscopically determine the position of the ear catheter during placement.

FIG. 8 is a detailed view of a first preferred embodiment of the distal section of the catheter tube 3, wherein the injection lumen 3A terminates in opening 3C at the distal tip of the atraumatic tip 12, and the pressure relief lumen 3B terminating in an opening 3D laterally offset at the atraumatic tip 12. The inflation lumen 3E terminates in the opening 3F in the area of the balloon 2.

Another preferred embodiment of the distal portion of the catheter tube 3 is shown in FIG. 9, wherein the pressure relief lumen 3B terminating laterally in an opening 3D distal to the balloon 2 but proximally to the tip (not shown). The inflation lumen 3E ends in the area of balloon 2 (the injection lumen is shown here).

FIG. 10 shows an insertion aid 101 according to the invention with an angled headpiece 102, a straight middle piece 103 and the end piece 104 split into two arms. Headpiece 102 forms an angle relative to the middle piece 103, ideally by appr. 70° (shown differently), i.e. said angle of 70° arising as a result of the run of the middle piece 103.

The end piece 104 is split so that it forms two arms, which serve as a handle for the treating physician to tear the insertion aid 101 open lengthwise. In continuation of the split section, a separator line or seam 105 extends along the length of the middle piece 103 and the end section 102, said line forming a weakening zone, for example by creating a partially incised area.

The insertion aid 101 is composed of a biocompatible polymer that has adequate rigidity as is needed to guide the system properly. At the same time, the polymer must not exhibit too much resistance which might otherwise impair tearing the insertion aid open. Customary polymers such as polyamide, polypropylene or copolymers such as Pebax are suitable.

FIG. 11 is a sequence of actions showing in image A the ear catheter 106 inserted through the nose of a patient by means of insertion aid 101 with the pressurizer 107 connected, in image B, the removal of the insertion aid 101 from the nose of the patient along the catheter tube, in image C, the insertion aid 101 already removed completely out of the patient's nose, in image D, the insertion aid 101 torn open to a large extent, in image E, the insertion aid 101 torn open up to the distal headpiece 102, and in image F, the insertion aid 101 which has been torn open completely and can now be disposed of.

FIG. 12 shows an insertion aid 101 proposed by the invention with the distal headpiece 102, the middle piece 103 and the two arms of the end piece 104 as well as the separator line 105 shown in broken lines, with the separator line 105 extending along the entire length of the middle piece 103 and the end piece 104. Said line starts at the junction point of the two arms of the end piece 104. It is understood that a corresponding separator line 105 is arranged on the opposite side.

FIG. 13 shows the proximal region of an inventive insertion aid 101 with a safeguard 108 arranged at the proximal end of the middle piece 103. Safeguard 108 consists of an adhesive medical strip that surrounds the insertion aid 101 at the location indicated and prevents premature tearing along the separator line 105. Before tearing open the insertion aid 101, the adhesive strip 108 is torn off by the attending physician.

In FIG. 14 a second embodiment of the insertion aid according to the invention is illustrated in the unfolded state, with a first half A comprising a half of the headpiece 102′, a half of the middle piece 103′ and a half of the end piece 104′ as well as a second half B comprising a half of the headpiece 102, a half of the middle piece 103 and a half of the end piece 104. In this example, the halves A, B of the insertion aid 101 are provided to form a mirror-inverted configuration identical to each other.

The headpiece 102, 102′ forms an angle relative to the middle piece 103, 103′, preferably by appr. 70° when used as an insertion aid for an ear catheter (shown differently here). The angle of 70° is formed based on the run/extension of the middle piece 103, 103′. An angle of the headpiece 102, 102′ relative to the middle piece 103, 103′ of 0° would thus result in an overall straight run of the middle and headpieces 103, 103′, 102, 102′.

In a region of the end piece 104, 104′, the halves A, B are connected to each other, preferably in a handle area 112, 112′, by means of a connecting element 111. The two end pieces 104, 104′, resp. the two parts of the handle area 112, 112′ are connected to one another preferably permanently by means of the connecting element 111, which, preferably, is already provided when the insertion aid 101 is manufactured, but alternatively it may be used to assemble the components later, i.e. the parts 104, 104′, respectively 112, 112′, or both halves A, B which initially may be manufactured as unconnected items.

A further connecting element 110A, 110B in the form of a latching component is additionally provided on end piece 104, 104′, with the interacting latching elements 110A, 110B each being arranged at the respective locations on the end piece 104, 104′.

A continuous duct extends through the insertion aid 101 through which a catheter can be passed. The duct gradually widens at the proximal end of the end piece 104, 104′ to facilitate insertion of the catheter (not shown) into the insertion aid 101.

FIG. 15 shows a perspective view of an alternative design of the second embodiment of the insertion aid 101 according to the invention, with this alternative design differing only with respect to the arrangement of the headpiece 102, which in the present case is not split into two halves but designed as an undivided tube. The headpiece is connected to the two-part middle piece 103, 103′ via a coupling element 109. The coupling element 109 is provided in the form of a sleeve that encompasses the proximal portion of the headpiece 102 and the distal portion of the middle piece 103, 103′. For example, force-fit plug-in connections or threads can be provided to join the headpiece 102, coupling element 109 and middle piece 103, 103′.

Thus, the coupling element 109 also serves as a further connecting element of the middle piece halves 103, 103′.

The remaining elements of the insertion aid 101, particularly the design of the end piece 104, 104′ with the further connecting element 110 corresponds to the second embodiment according to FIG. 14.

FIG. 16 is a top view of the inside of one half of the insertion aid 101 as per FIG. 15, as a longitudinal section. The individual elements correspond essentially to FIG. 14, to which reference is made as appropriate. This representation of the alternative design however differs in that in particular the arrangement of the coupling element 109 is shown, which is provided here as a sleeve embracing the adjoining ends of the headpiece and middle piece.

FIG. 17 shows in detail partial views of certain portions of the alternative second embodiment shown in FIGS. 15 and 16.

FIG. 17A shows a longitudinal section through the distal end of the headpiece.

FIG. 17B shows the coupling element 109 in longitudinal section with the respective ends of the headpiece 102 and the middle piece 103. These three elements are connected via threads, i.e. a female thread in coupling element 109 and male threads on the relevant areas of the headpiece 102 and the middle piece 103.

Finally, in FIG. 17C the proximally widening portion of the end piece 104 can be seen which is provided to facilitate introducing a catheter (not shown) into the insertion aid.

FIG. 18 illustrates a perspective view of the alternative design of the second embodiment of the insertion aid 101 according to the invention as shown in FIG. 15. Additionally, this embodiment includes a sheath 113 provided on and closely fitting the head and/or middle piece 102, 103. Sheath 113 provides additional support to the two-piece head and/or middle pieces 102, 103 by holding the two halves of these components together.

FIG. 19 shows a detail of a section of the sheath 113. A perforation line 114 may be provided to enable opening and removal of the sheath 113. However, such a perforation is mainly for operational convenience and may be omitted without any problems. Alternatively, the user may elect to remove the sheath by making a longitudinal cut or by tearing it open.

### LIST OF REFERENCE NUMERALS

1 Ear catheter
2 Shield (A: distal section; B: middle section; C: proximal section)
3 Catheter tube (A: injection lumen; B: pressure relief lumen; C: distal opening of the injection lumen; D: distal opening of the pressure relief lumen; E: inflation lumen; F: distal opening of the inflation lumen)
4 One-way valve
5 Connection device (Luer-Lock system)
6 Expansion device
7 Operating aid
8 Latching device
9 Connector
10 Proximal connections (A: injection lumen; B: pressure relief lumen; C: inflation lumen)
11 Marker (A: first marker; B: second marker; C: third marker)
12 Atraumatic tip
101 Insertion aid
102 Headpiece (102 first half, 102′ second half)
103 Middle piece (103 first half, 103′ second half)
104 End piece (104 first half, 104′ second half)
105 Separator line
106 Catheter
107 Pressurizer
108 Safeguard
109 Coupling element
110 Further connecting element (110A, 110B sections of further connecting element)
111 Connecting element
112 Handle area (112 first part, 112′ second part)
113 Sheath
114 Perforation line
d distal
p proximal

1. Ear catheter for insertion into the eustachian tube, including a first tube as catheter tube (3) with at least one first lumen as injection lumen (3A) for application of a liquid, at least one second lumen as pressure relief lumen (3B) for pressure relief, and a self-expandable sealing element (2) for the occlusion of the eustachian tube.

2. Ear catheter according to claim 1, characterized in that the sealing element (2) is provided in the form of a shield at the distal end of the catheter tube (3) and an expansion device (6) is provided for controlling the expansion of the shield (2).

3. Ear catheter according to claim 2, characterized in that the shield (2) comprises self-expandable support elements and/or a self-expandable framework and a membrane secured thereto.

4. Ear catheter according to claim 3, characterized in that the self-expandable support elements and/or the self-expandable framework are made of a shape memory material.

5. Ear catheter according to claim 3, characterized in that the membrane is made of PTFE or ePTFE.

6. Ear catheter according to claim 2, characterized in that the expansion device (6) is provided as a second tube having a distal and a proximal end, said tube being provided in a form-closed manner externally around the catheter tube (3) and longitudinally displaceable relative to the catheter tube (3), and wherein a displacement of the expansion device (6) in proximal direction causes the shield (2) to be released for expansion and a distal displacement results in a compression of the shield (2).

7. Ear catheter according to claim 6, characterized in that the expansion device (6) comprises an operating aid (7) arranged at its proximal end.

8. Ear catheter according to claim 1, characterized in that the sealing element (2) is provided as a balloon at the distal end of the catheter tube (3) and the catheter tube (3) comprises a further lumen as inflation lumen (3E).

9. Ear catheter according to claim 1, characterized in that the catheter tube (3) is provided with at least one connection device (5), in particular a Luer-Lock system (5), which is located at its proximal end.

10. Ear catheter according to claim 1, characterized in that the injection lumen (3A) and the pressure relief lumen (3B) extend proximally from the connection device to distally of the sealing element (2).

11. Ear catheter according to claim 1, characterized in that the distal end (12) of the ear catheter (1) is designed so as to be atraumatic.

12. Ear catheter according to claim 1, characterized in that at least one one-way valve (4) is provided in at least one of the lumens of the catheter tube (3), in particular in the injection lumen (3A) or in the pressure relief lumen (3B).

13. Ear catheter according to claim 1 provided with an insertion aid, characterized in that the insertion aid (101) comprises a headpiece (102, 102′), a middle piece (103, 103′) and an end piece (104, 104′), wherein the insertion aid (101) consists of a biocompatible polymer, and at least one separator line (105) extends at least through the middle piece (103, 103′) and the end piece (104, 104′) each, which enables the insertion aid (101) to be unfolded or split open along said line.

14. Insertion aid in particular for an ear catheter according to claim 1 provided with a headpiece (102, 102′), a middle piece (103, 103′) and an end piece (104, 104′), wherein the insertion aid (101) consists of a biocompatible polymer, characterized in that at least one separator line (105) extends at least through the middle piece (103, 103′) and the end piece (104, 104′) each, which enables the insertion aid (101) to be unfolded or split open along said line.

15. Insertion aid according to claim 14, characterized in that at least the middle piece (103, 103′) and the end piece (104, 104′) of the insertion aid (101) are provided divided into two longitudinal parts (A, B) as a result of two oppositely located separator lines (105).

16. Insertion aid for catheters according to claim 14, characterized in that the headpiece (102) of the insertion aid (101) is provided in a one-piece tubular form and can be connected at its proximal end to the distal end of the middle piece (103, 103′) by means of a coupling element (109), wherein the middle piece (103, 103′) and the end piece (104, 104′) forming a basic element.

17. Insertion aid for catheters according to claim 14, characterized in that the end piece (104, 104′) comprises a connecting element (111) by means of which the two longitudinal parts (A, B) are connected to each other.

18. Insertion aid for catheters according to claim 17, characterized in that the end piece (104, 104′) comprises a further connecting element (110), which is preferably arranged on the opposite side of the connecting element (111) and detachably connects the two longitudinal parts (A, B) to each other.

19. Insertion aid for catheters according to claim 13, characterized in that two continuous separator lines extend through the headpiece (102, 102′) and the middle piece (103, 103′) along which the insertion aid (101) can be split up, and with the end piece (104, 104′) being split in two in continuation of these separator lines (105).

20. Insertion aid according to claim 14, characterized in that a safeguard (108) is provided at the proximal end of the middle piece (103).

21. Insertion aid according to claim 13, characterized in that a tubular sheath (113) is provided around the middle piece (103) and/or the headpiece (102).
